(19) United States
(12) Patent Application Publication
(10) Pub. No.: US 2024/0417769 A1
(43) Pub. Date: Dec. 19, 2024

(54) POLYNUCLEOTIDE SYNTHESIS METHOD AND SYSTEM THEREFOR

(71) Applicant: Massachusetts Institute of Technology, Cambridge, MA (US)

(72) Inventors: Nikhil Lal, Cambridge, MA (US); Eyal Perry, Cambridge, MA (US); Joseph Jacobson, Cambridge, MA (US)

(73) Assignee: Massachusetts Institute of Technology, Cambridge, MA (US)

(21) Appl. No.: 18/745,495

(22) Filed: Jun. 17, 2024

Related U.S. Application Data
(60) Provisional application No. 63/508,713, filed on Jun. 16, 2023.
(60) Provisional application No. 63/512,787, filed on Jul. 10, 2023.

(51) Int. Cl. C12P 19/34; B01J 19/00

(52) U.S. Cl. CPC C12P 19/34; B01J 19/0046; B01J 2219/00495; B01J 2219/00596; B01J 2219/00722

(57) ABSTRACT

A method of polynucleotide synthesis includes a series of enzymatic reactions for restriction, ligation, and amplification using a machine and system that provide efficient droplet-based reactions. The method enables hierarchical assembly with minimal handling in order to address challenges associated with traditional polynucleotide synthesis methods, such as high cost, limited throughput, long synthesis times, and limited ability to synthesize long sequences.

### CROSS-REFERENCE TO RELATED APPLICATION

This application claims the benefit of priority of U.S. provisional application No. 63/508,713, filed Jun. 16, 2023, and U.S. provisional application No. 63/512,787, filed Jul. 10, 2023. The contents of both application No. 63/508,713 and 63/512,787 are herein incorporated by reference in their entireties.

### BACKGROUND OF THE INVENTION

The present invention relates to systems and methods for synthesizing nucleotides and, more particularly, to systems and methods that incorporate state-of-the-art inkjet printing principles for synthesizing unique nucleotides.

The artificial construction of Deoxyribonucleic acid (DNA) or Ribonucleic acid (RNA) sequences has emerged as a pivotal technology in modern biotechnology and pharmacology/pharmaceutical manufacturing. These constructs play a crucial role in the fields of genetic engineering, biological engineering, and therapeutics. Today, the longest standard sequence length available from a commercial source is about 1.8 kbp, yet the shortest synthetic genome is about 160 kbp. Consequently, the bulk of synthetic biology is limited in the fraction of an organism's genome that can be modified at reasonable cost. The challenges and costs associated with manual gene-editing processes present a significant burden for biotechnological innovation, and an opportunity for improvement.

Current polynucleotide/gene synthesis technologies (GST) face significant barriers preventing synthesis of longer fragments. Despite drastic reduction in cost per base pair (bp) in oligonucleotide synthesis, the same cannot be said for the synthesis of long polynucleotides/gene fragments. The current gold standard for oligonucleotide synthesis uses phosphoramidite chemistry for the assembly of single-stranded DNA (ssDNA) which has the dual disadvantage of high error rates and significant costs, preventing efficient implementation. The high error rates are due to difficulties in handling the ssDNA as the length of the oligomer synthesis exceeds 200 bp, e.g., self-ligation or hairpin formation, and unpredictability of the phosphoramidite chemistry.

In synthesizing longer polynucleotide sequences, the likelihood and impact of errors in assembly increase dramatically with the length of sequence.

As can be seen, there is a need for double strand DNA (dsDNA)-synthesis methods for production of nucleotides having a length in excess of 1.8 kbp while mitigating the costs and challenges associated with phosphoramidite-chemistry syntheses.

### SUMMARY OF THE INVENTION

In one aspect of the present invention, a method of assembling polynucleotides comprises providing a reaction apparatus including a first substrate (90) with a first surface (80) having an acceptor DNA fragment (1) bound thereon at points in an array, and a second substrate (90) with a second surface (80) having a donor DNA fragment (170) bound thereon at points in a corresponding array, with the first surface (80) aligned against the second surface (80) such that the first and second substrates (90) form a reaction volume therebetween; applying an enzyme mixture (100, 180, 190) as droplets to the first surface and the second surface (80), thereby: cleaving (110) the acceptor and donor DNA fragments (1, 170) at an enzyme recognition site (30, 50, 150) with the enzyme mixture (100, 180, 190) to liberate at least one donor sequence (160) having a first single strand free end (210) and to expose at least one acceptor sequence (40) having a second single strand free end (120) that is complementary to the first single strand free end (210); and ligating (200) the first single strand free end (210) to the second single strand free end (120) to form an intermediate DNA fragment (1) having a sequence (230) concatenated from the donor sequence (160) and the acceptor sequence (40), wherein the intermediate DNA fragment (1) is bound to the first surface or the second surface (80); and washing (130) the first surface (80) and the second surface (80) to remove the enzyme mixture (100, 180, 190) and unbound byproducts (10, 20, 30, 220).

In another aspect of the present invention, a nucleotide assembly reaction apparatus comprises a first substrate (510) having a first planar surface with a first array of reaction spots (515) operative to bind an individual nucleotide chain; and a second substrate (550) having a second planar surface with a second array of reaction spots operative to bind an individual nucleotide chain, having a reaction position facing the first planar surface. The first planar surface and the second planar surface form a plurality of individually addressable, reversibly enclosable reaction chambers (570) therebetween.

The polynucleotide synthesis method disclosed herein uses a series of enzymatic reactions and controlled-droplet management, which mitigate the costs and challenges associated with phosphoramidite chemistry synthesis and enable synthesis of DNA strands in excess of 1.8 kbp.

One aspect of the present subject matter is directed to methods and systems for assembling preselected oligonucleotides and polynucleotides. A further aspect of the present subject matter is directed to methods and systems that incorporate state-of-the-art inkjet-printing principles for purposes of synthesizing a variety of preselected, and certain unique, polynucleotides.

The present subject matter thus addresses challenges associated with traditional polynucleotide synthesis methods and systems including but not limited to high costs, throughput restrictions, long and costly synthesis times, and limited ability to synthesize longer sequences. The present disclosure includes a) a series of enzymatic reactions for restriction, ligation, and amplification; b) a machine operation methodology and tool set for efficient droplet-based reactions; and c) a system of hierarchical assembly which includes minimal handling steps in order to address current limitations in the field.

Features of the present subject matter include individually addressable virtual micro-wells on opposing planar surfaces; plate to plate (donor to acceptor) transfer by iterative cutting and ligation; individually addressable reversibly enclosable reaction chambers; hierarchical and/or multiplexed assembly with pre-sequenced oligonucleotide (small) parts; assembly with enzymatically cleavable and ligatable capping groups (i.e., forming “dogbones”); and assembly with in-situ amplification (nick translation amp), wherein “getters”/“grabbers” attach the amped product to the plate to enable a wash step.

These and other features, aspects and advantages of the present invention will become better understood with reference to the following drawings, description and claims.

### BRIEF DESCRIPTION OF THE DRAWINGS

FIG. 1 is a schematic view of a polynucleotide assembly process according to an embodiment of the present invention, showing acceptor deprotection;

FIG. 2 is another schematic view thereof, showing a donor assembly restriction step;

FIG. 3 is another schematic view thereof, showing a donor assembly ligation step;

FIG. 4 is another schematic view thereof, showing a donor assembly washing step;

FIG. 5 is another schematic view thereof, showing an isothermal donor assembly Strand Displacement Amplification (SDA) step;

FIG. 6 is another schematic view thereof, showing an isothermal donor assembly Nicking Enzyme Amplification Reaction (NEAR) step;

FIG. 7 is another schematic view thereof, showing an isothermal donor assembly Hybridization step;

FIG. 8 is another schematic view thereof, showing an isothermal donor assembly washing step;

FIG. 9 is a schematic view of a thermocycle donor amplification solid-phase polymerase chain reaction (SP-PCR) step;

FIG. 10 is another schematic view thereof, showing a thermocycle donor amplify annealing step;

FIG. 11 is another schematic view thereof, showing a thermocycle donor amplify extension step;

FIG. 12 is another schematic view thereof, showing a thermocycle donor amplify denaturation step;

FIG. 13 is a schematic view of an alignment and ligation assembly method according to an embodiment of the present invention;

FIG. 14 is another schematic view thereof, showing assembly of a higher order polynucleotide;

FIG. 15 is another schematic view thereof, illustrating separation of DNA fragments;

FIG. 16 is a front elevation schematic view of a system according to an embodiment of the present invention, showing a series of method steps;

FIG. 17 is another front elevation schematic view thereof;

FIG. 18A is a schematic view of a system according to an embodiment of the present invention, showing a first means of reducing droplet dry-out;

FIG. 18B is another schematic view thereof, showing a second means of reducing droplet dry-out;

FIG. 18C is another schematic view thereof, showing a third means of reducing droplet dry-out;

FIG. 18D is another schematic view thereof, showing a fourth means of reducing droplet dry-out;

FIG. 18E is another schematic view thereof, showing a fifth means of reducing droplet dry-out;

FIG. 18F is another schematic view thereof, showing a sixth means of reducing droplet dry-out;

FIG. 19 is a top plan schematic view of a system according to an embodiment of the present invention, illustrating a series of steps using two plates;

FIG. 20 is a flowchart of a hierarchical assembly method according to an embodiment of the present invention;

FIG. 21 is a front elevation view of a system according to the present invention, showing a harvesting method step; and

FIG. 22 is a schematic view of a click reaction applied in the method of FIG. 16.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method and system that automate a series of enzymatic biochemical reactions using a unique combination of known biological compositions-of-matter with a so-called “plate-to-plate” implementation. Genes may be assembled by transferring and joining short gene fragments (oligonucleotides and polynucleotides) between fixed substrates in micro-reaction vesicles. An enzyme first cuts one section of a gene fragment, the payload, and ligates that section to the opposing plate. The steps are repeated until the target construct, greater than 1.8 kbp, is synthesized.

The complementary gene fragments may be referred to as a donor and a receptor and the resulting construct may be referred to as a product. For example:
Acceptor (k+4 bases): 5′-N^{k}N₁N₂N₃N₄-3′
Donor (k+4 bases): 5′-N₁N₂N₃N₄N^{k}-3′
Product (2k+4 bases): 5′-N^{k}N₁N₂N₃N₄N^{k}-3′
where k is the number of bases.

As used herein, the term “dogbones” refers to a double stranded oligonucleotide sequence having enzymatically cleavable and ligatable capping groups.

As used herein, the term “hairpin” refers to oligonucleotide sequences containing a functional group that covalently binds to the surface of a glass slide or substrate; i.e., a sticky end, that is complementary to the sticky end of a target fragment.

The term a “perfect part” is defined herein as either a) an on-demand, sequence-verified polynucleotide sequence, or b) an oligonucleotide selected from a library of parts that have been previously sequenced and produce predictable results such that they can be considered trusted sequences.

The term “grabbers” as used herein refers to a ssDNA sequence that is complementary to the terminal sequence of a synthesized hairpin. The hairpin hybridizes to the grabber, retaining amplified product on a substrate surface while the enzymes and primers are washed away.

The term “click chemistry” refers to a chemical philosophy introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. A distinct exothermic reaction makes a reactant “spring loaded”. In some embodiments, a click chemistry reaction exhibits high atom economy, can be carried out under simple reaction conditions, uses readily available starting materials and reagents, uses no toxic solvents or uses a solvent that is benign or easily removed (preferably water), and/or provides simple product isolation by non-chromatographic methods (crystallization or distillation).

The terms “substrate”, “slide”, and “plate” are used interchangeably herein. The term “plate” is generally used to emphasize substrate-agnostic DNA assembly processes. A substrate having polynucleotides bound thereon is sometimes referred to herein as a “chip”. Glass slides are discussed in some examples that may have surface chemistry specific to a glass substrate. However, the present invention is not limited to slides, plates, or glass substrates. Any suitable substrate having any suitable configuration may be used.

As used herein, the term “star activity” refers to altered specificity of some restriction enzymes, which may cleave sequences that are not identical to their recognition sequence under non-standard reaction conditions.

The term “template strand” as used herein refers to the initial nucleotide sequence that is amplified as disclosed herein. The term “coding strand” is used herein to refer to a duplicate of the template strand produced by the amplification process.

As used herein, polynucleotides are referred to as “first order” with respect to initial assembly steps that produce a concatenated sequence and as “higher order” with respect to subsequent assembly steps.

Polynucleotide constructs/gene fragments are designed around specific sequences that enable particular enzymatic reactions selected for the method steps disclosed herein. Every construct/fragment has a unique recognition sequence for each cut site, for overlap sites (i.e., sticky ends for ligation), and for the payload itself. Each polynucleotide fragment is capped on both ends with “hairpin” structural configurations. At least one of these hairpins contains a functional group that is covalently bound to the glass slide using a Click chemistry. The other hairpin serves to prevent mis-ligation during the reaction. The initial oligonucleotide constructs are “spotted” across the glass slide in a precision array using an inkjet head or a spotting pin.

For example, to assemble a 100 kbp strand of DNA starting from single target nucleotide assemblies, and conducting one ligation step per assembly step, a system may have a minimum of 2¹⁷ individual spots. If each substrate can hold an array of 2⁹×2⁹ spots, for a total of 2¹⁸ spots, the strand may be fully assembled with plate-to-plate contact and efficient path planning. The system can synthesize one assembly or many assemblies of different lengths simultaneously. In the example substrate discussed supra, 2¹⁸ distinct assemblies may be maximally assembled from each plate, using a catalog of plates with complementary spots. In an example, to produce an assembly having a length of, e.g., about 100 kbp by a method having 18 assembly steps, the system may comprise at least 2¹⁸ plates. However, in practice, plates can support multiple spots related to the same assembly, making it efficient to mix spots for simultaneous assemblies. A human genome is 3e⁹ bp in length. In another example, if a single ligation occurs in each step, 3e⁹ spots are generally believed necessary to assemble a full genome, one for each starting nucleotide. If each substrate has a 2¹¹×2¹¹ array of droplets with 10 pL spot volumes, i.e., greater than 2³² bp, the system may have 2¹⁰ substrates.

The enzymatic reactions are performed utilizing precise amounts of predetermined biochemical reagents present between the glass plates to form the reaction vesicle. A deposition tool like an inkjet print head or a single nozzle droplet ejector containing a mixture of cutting enzymes and ligation enzymes deposits droplets, on the order of 1 nL, in precise alignment with the spotted array of polynucleotide constructs. To mitigate a substantial change in reaction composition due to evaporation, the glass slides are rapidly oriented with precision alignment of the respectively spotted arrays. The slides are brought into close contact: e.g., for a 1 nL reaction volume the slide surfaces may be spaced 50 μm apart. This results in a characteristic reaction dimension of about 160 μm in diameter. The diameter of the reaction dimension is thus generous enough to overlap spots placed on opposing slides and to account for any minor misalignment between the slides.

Without a barrier, the droplets are prone to “dry-out”, e.g., by evaporation. A barrier layer, e.g., oil, may be added to both substrates to mitigate dry-out. In some cases, the process may be carried out in a pressurized gas chamber, or the entire machine may be pressurized. Alternatively, or in addition, humidity of the glass slide surface and/or the machine as a whole may be precisely controlled. In some embodiments, porous glass substrates may be used to introduce humidity into the array on the slide surface. The latter approach is believed to be especially useful for high temperature reactions where humidified air from droplet evaporation may rapidly leave the reaction area. If ambient conditions are exceptionally dry, the substrates may have etched nanofluidic pipes that refill each droplet in an array.

Notably, a membrane or filter may be used to separate DNA from flowing backwards through the channel and cross-contaminating another point on the array. This membrane may be actively or passively controlled, as in the case of an ionomer membrane that can be electrically modulated to open and close the channel to DNA or other charged molecules in solution.

Within the reaction volume, a first operation uses a single restriction endonuclease enzyme to cut polynucleotide fragments on both plates. Suitable enzymes may include but are not limited to, for example BsaI-HF, a high-fidelity enzyme derived from Bacillus thermophilus, BbsI-HF, a high-fidelity enzyme derived from an E. coli strain that carries the cloned BbsI gene from Bacillus brevis, or BsmBI, a Type IIS restriction endonuclease. This exposes complementary 4-nucleotide sticky ends on each fragment. The cut site for the construct on one plate liberates the payload, and the cut site on the opposing plate removes a hairpin that previously capped the target sticky end. Given sufficient time, the sticky end from the payload finds the sticky end on the target plate, and a ligation enzyme present in the reaction vesicle joins the payload to the target plate. This reaction occurs simultaneously across the plate where each spot may uniquely serve as a donor or acceptor. This process repeats itself across all spots by moving the plates in a predetermined pattern that results in a hierarchical assembly of these donor/acceptor reactions. Once a spot has donated its payload, that spot no longer participates in this repeated process. To ensure that a spot may donate or accept, every construct has two distinct cut sites, one for its activation as a donor and the other for its activation as a receiver. For each construct to find its respective complementary target on a plate, the cut sites are inverted, e.g., a BsaI-HF cut site on the base of a first construct correlates to de-capping on the complementary construct, while a BbsI-HF cut site on the base of the complementary construct correlates to de-capping on the first construct.

Subsequent to every reaction between a set of spots, a washing process is performed that neutralizes the surface, cleans active reagents, and removes excess polynucleotides. After spotting is completed, a deposition tool returns to re-populate the target spots with the appropriate reagents. At the end of the assembly process, the fully assembled polynucleotides are harvested using a syringe that first deposits the final cutting enzyme in a droplet to liberate assembled polynucleotides from the plate, and then aspirates the liberated polynucleotides from within the droplet. The total droplet volume may be minimized to reduce dilution of the finished assembly.

The foregoing disclosure describes a novel method of polynucleotide synthesis. To create a useful polynucleotide construct of significant length and minimize the error rate, the input oligomers used for assembly should be so-called “perfect parts”, e.g., the reagents are known and/or verified prior to assembly. Compared to phosphoramidite polynucleotide synthesis, the unit operations of enzymatic assembly have a lower likelihood of misligation, so the most likely source of error, especially when assembling larger fragments, is from the input sequences. “Perfect parts” as an input to the hierarchical assembly process can mitigate this source of error.

To enable a “perfect part” assembly system, there are two key paths that can be followed: a) during the assembly process after a ligation step, a selection of polynucleotides may be harvested and sequence verified; and/or b) all oligonucleotide sequences used as inputs to initial assembly may be considered as part of a known-good library. In both instances, the finished good assemblies should, under nominal conditions, precisely match the sum of payload sequences of the inputs. By using a combination of double strand DNA fragments and existing high efficiency ligation enzymes, e.g., T4 DNA ligase, overlapping matched sticky ends of said fragments are ligated with high expected accuracy.

Subsequent to verification, polynucleotides are bound to the substrate in preparation for hierarchical assembly. Following one or more assembly steps, these constructs can be harvested and sequenced to verify successful assembly and to ensure a minimal rate of misligation. In some instances, it is possible to successfully harvest a reduced quantity of the newly formed polynucleotides so as not to prevent continuation of subsequent assembly steps. In other instances, it will be beneficial to harvest all assembled polynucleotides, sequence and verify the parts, and bind them to a new substrate in preparation for higher order assembly.

A library of oligonucleotide sequences, or parts, may be either single stranded DNA, double stranded DNA, or some mixture of the two based on the structure of the sequence, e.g., a hairpin. The parts are verified as being “perfect” prior to being stored as either identical or diverse arrays on a multi-plate or multi-slide storage system. Plates or slides are pulled on demand from the storage system and used to assemble constructs in a hierarchical polynucleotide synthesis method. The parts may be selectively harvested and bound to new slides prior to assembly, or they may be used as-is in their stored condition. Parts are then used in the previously described hierarchical polynucleotide synthesis method.

In some embodiments, the method may have as few as three hierarchical assembly steps with DNA bases and enzymes.

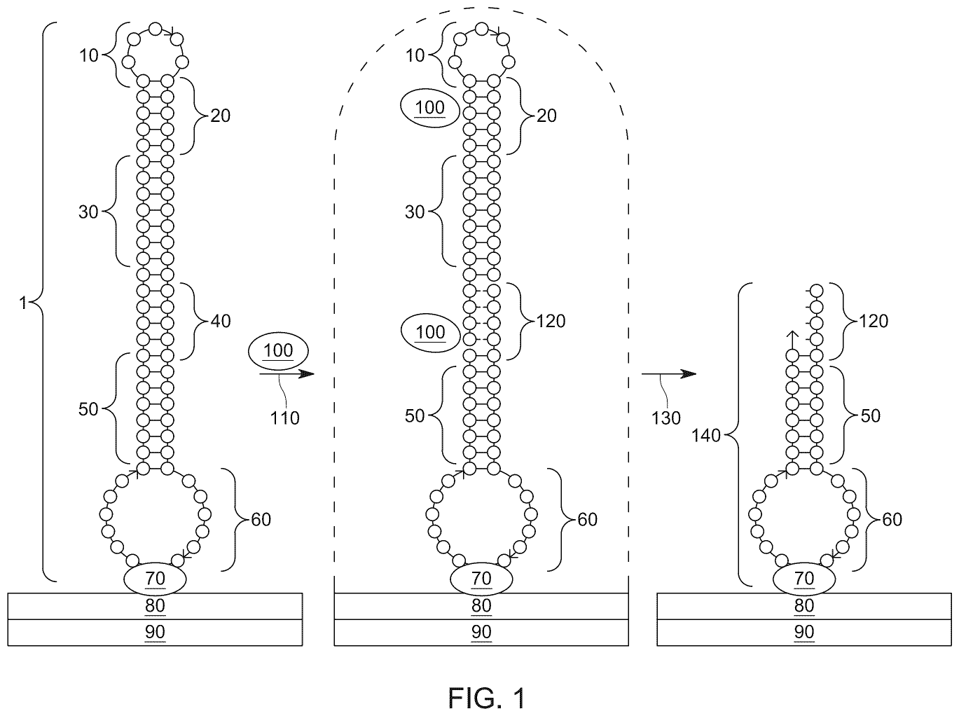

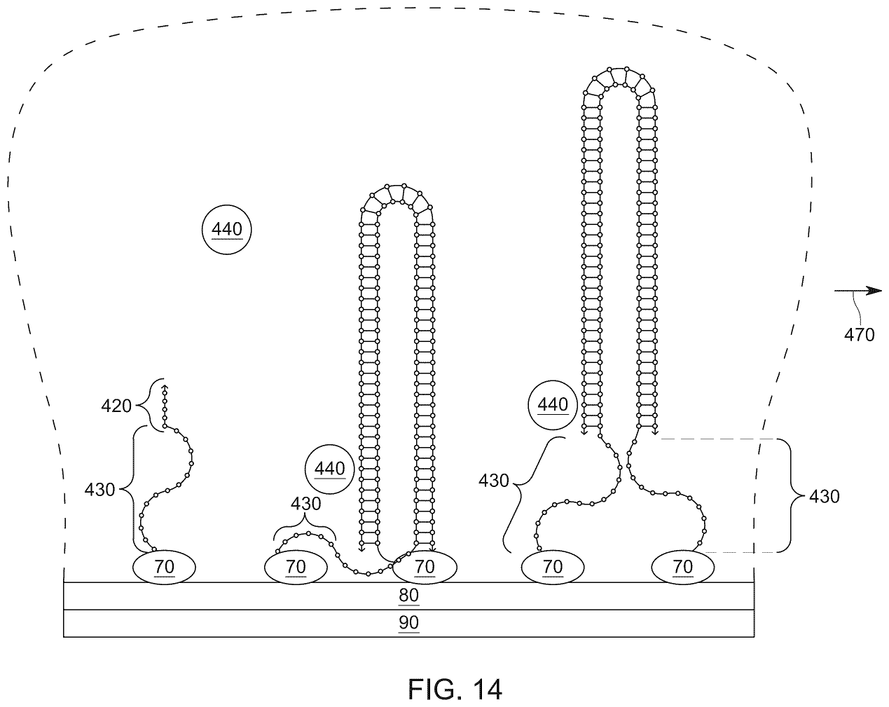

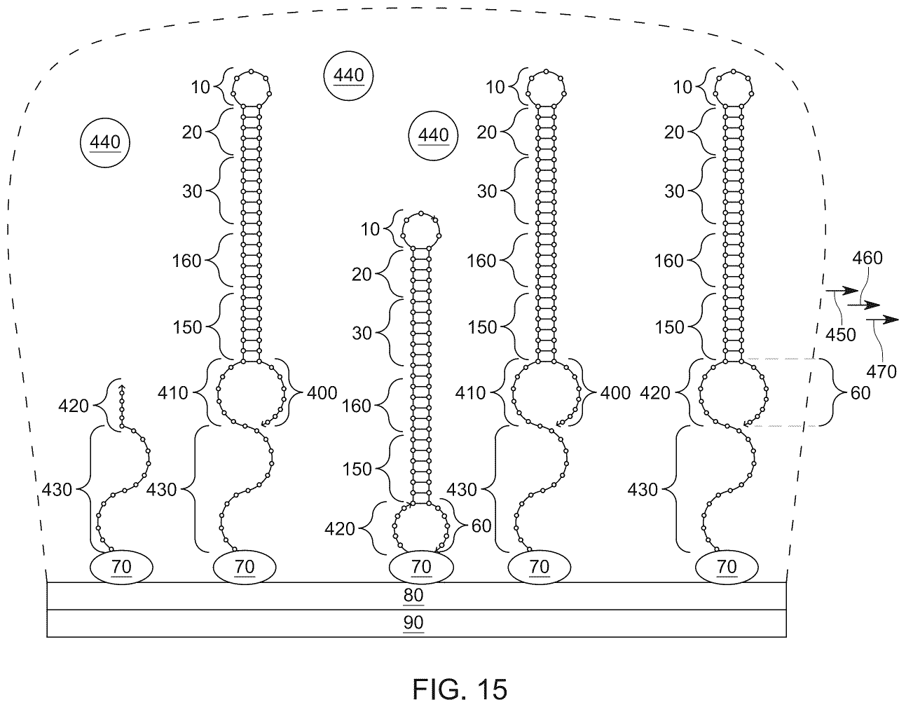

Turning now to FIGS. 1-17, FIGS. 18A, 18B, 18C, 18D, 18E, and 18F, and FIGS. 19-22, a three-step hierarchical assembly process includes deprotecting an acceptor, binding the acceptor with a donor, and amplifying the donor to produce a product.

FIG. 1 illustrates a deprotection process according to an embodiment of the present invention. As shown in FIG. 1, a substrate 90, such as a glass slide, has a coating 80 on one surface effective for DNA attachment, e.g. a composition containing azide groups. Immobilized on the surface 80 is a strand or DNA fragment 1 having a generally dogbone-shaped configuration, comprising a double stranded stem with a single loop 10, 60 at each end. The double stranded stem generally comprises a stem 20 having a length of e.g., about 20-30 bp; a Type IIS Restriction Endonuclease (e.g., BbsI) Recognition Site 30; a unique Acceptor Payload Sequence 40, having a length of about k+4, e.g. ACTGG/CCAGT (k=1); and a Type IIS Restriction Endonuclease (e.g., BsaI) Recognition Site 50. The top loop 10, which may have a length of e.g., about 4-8 bp, acts as a capping group. The bottom loop 60 is a single stranded primer binding sequence or priming site for Donor Amplification e.g., by Copper-free Click chemistry, generally having a length of about 20 bp, attached to the functionalized surface 80 by an internal DNA modification 70, e.g. by internal Dibenzocyclooctyl [DBCO] modification. The recognition site 30 may be, for example, TCGTCTTC/GAAGACGA. The recognition site 50 may be, for example, GGTCTCG/CGAGACC. The immobilized strand 1 may be developed to either an acceptor or donor in a deprotection step, wherein a fragment is uncapped with a Type IIS restriction enzyme 100 (e.g., BbsI) reaction via a digestion step 110. The enzyme 100 cleaves the top loop 10 and short stem 20, 30 from the strand, leaving a sticky 5′ end 120 e.g., CTGG, of the acceptor payload sequence 40. The surface 80 is washed 130 to remove the enzyme 100, leaving immobilized fragments with a single loop 60, a double stranded stem 50, and the sticky end 120. To fabricate fragments 1, universal DBCO groups 70 can be attached on the surface 80 and ligated with DNA hairpins 140 that carry unique payload sequences. For example, the acceptor may be 5′-ACTGG-3′; the donor may be 5′-CTGGT-3′; and the product may be 5′-ACTGGT-3′.

In the deprotection process, a MutS error correction enzyme or similar can be used to disable fragments that have been misligated in previous steps.

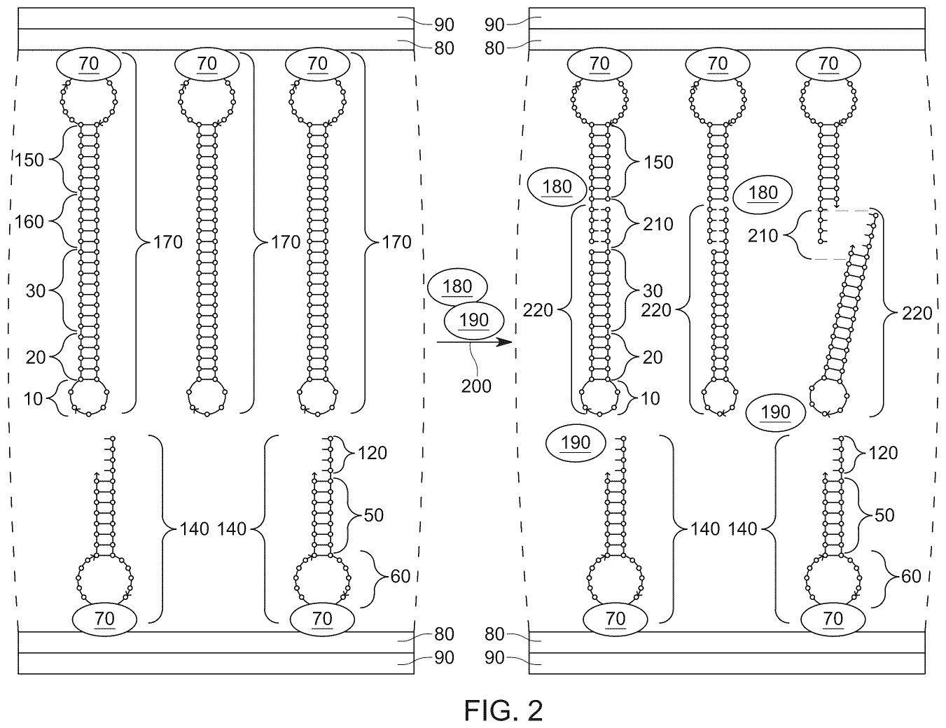

Turning to FIG. 2, an oligonucleotide assembly method includes reactions performed between acceptor and donor chips. The deprotected acceptor chip contains DNA hairpins 140 attached to the surface with sticky ends 120 extending therefrom. The donor chip contains capped DNA donor strands 170 containing the Donor Payload Sequence 160 (e.g., CTGGT/ACCAG with a length of k+4, where k=1). See FIG. 2. First, a type IIS restriction enzyme 180 (e.g., BsmBI) and DNA ligase 190 are added to a reaction volume that is encapsulated between the chips. The restriction enzyme 180 cleaves the fragments on the donor surface that have a unique recognition site 150, e.g., BsmBI (Restriction Endonuclease Type IIS). As a result, “floating” DNA hairpins 220 with a sticky 5′ end 210 (e.g., CCAG) are suspended in liquid phase between the two chips.

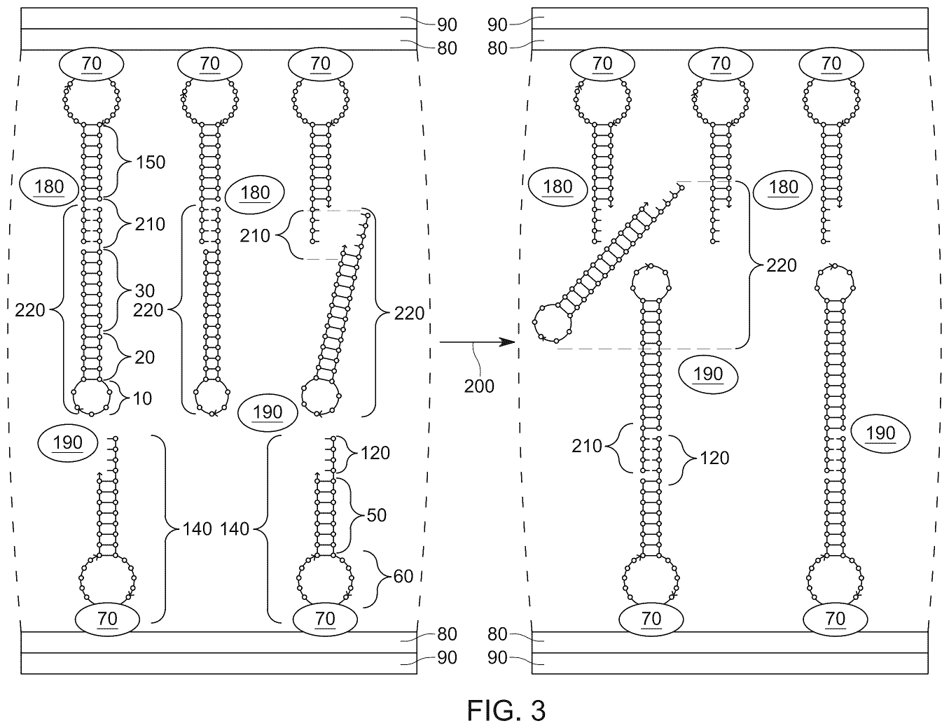

Next, the floating DNA hairpins 220 can either bind to the uncapped DNA hairpins on the donor chip or the acceptor chip, catalyzed by DNA Ligase 190. If the hairpins 220 bind back on the donor chip, the restriction enzyme 180 cleaves the bond. If the hairpins 220 bind to the acceptor chip, no further cleaving occurs. Sticky end 210 is precisely complementary to the sticky end 120 on the acceptor fragment, as shown in FIG. 3.

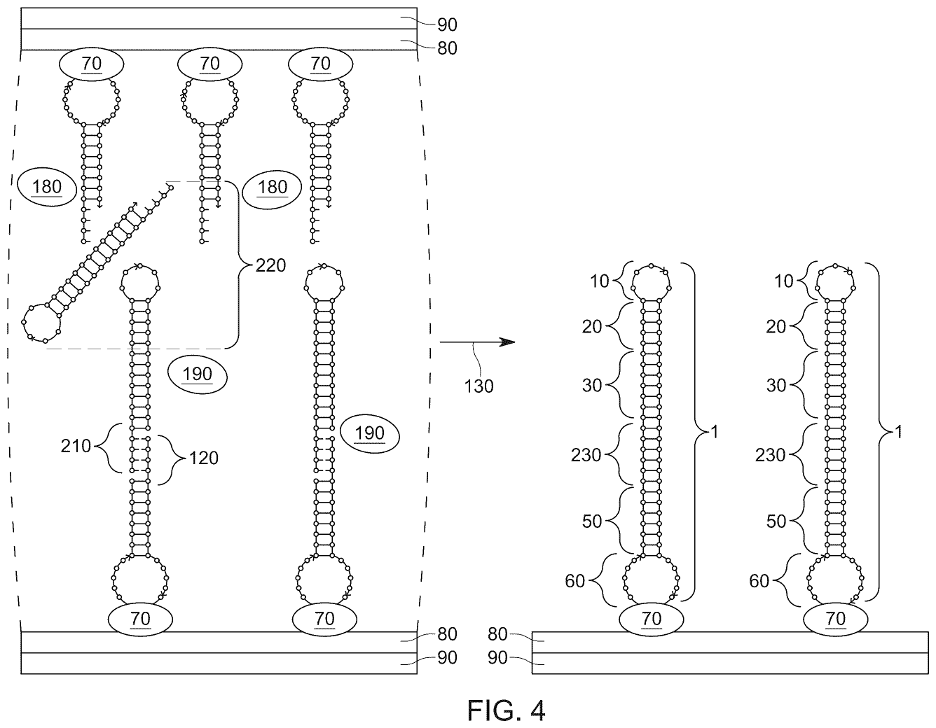

Finally, the reaction enzymes 180, 190 are washed 130. The result, shown in FIG. 4, is an acceptor chip that contains capped DNA fragments 1 with “perfect” payload sequences which are a concatenation of the donor sequences and the acceptor sequences, shown as a joined region 230 comprising sticky ends 120 and 210. In an example, the payload sequence, may have a length of 2k+4 where k=1, e.g., ACTGGT/ACCAGT.

The acceptor chip can be used in later steps either as a donor or acceptor. Further steps can be taken to ensure that fragments that failed to go through ligation 200 are disabled from future assembly. Running an exonuclease reaction enables any uncapped fragments to be chewed back, rendering them disabled for further assembly steps. Alternatively, suspending an excess of capping molecules on the chip, e.g. DNA hairpins with 4 degenerate base sticky ends (that can bind to any sequence) along with DNA Ligase, will cap the remaining fragments.

A donor chip may be prepared by amplification to ensure an excess of donor fragments over acceptor fragments. Two paths for on-chip amplifications of DNA fragments are described below.

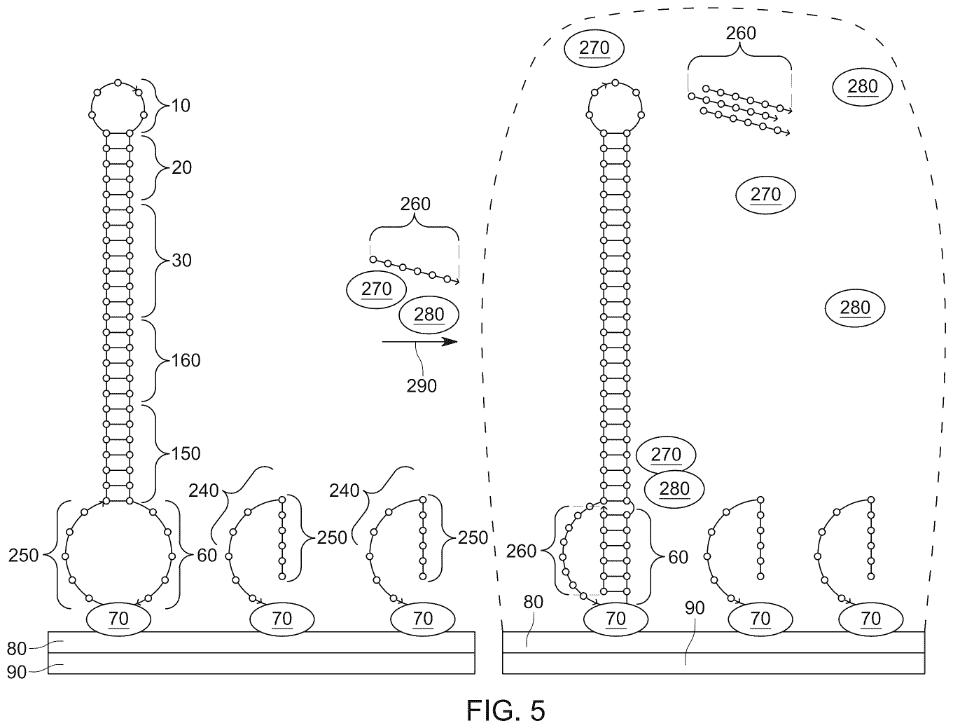

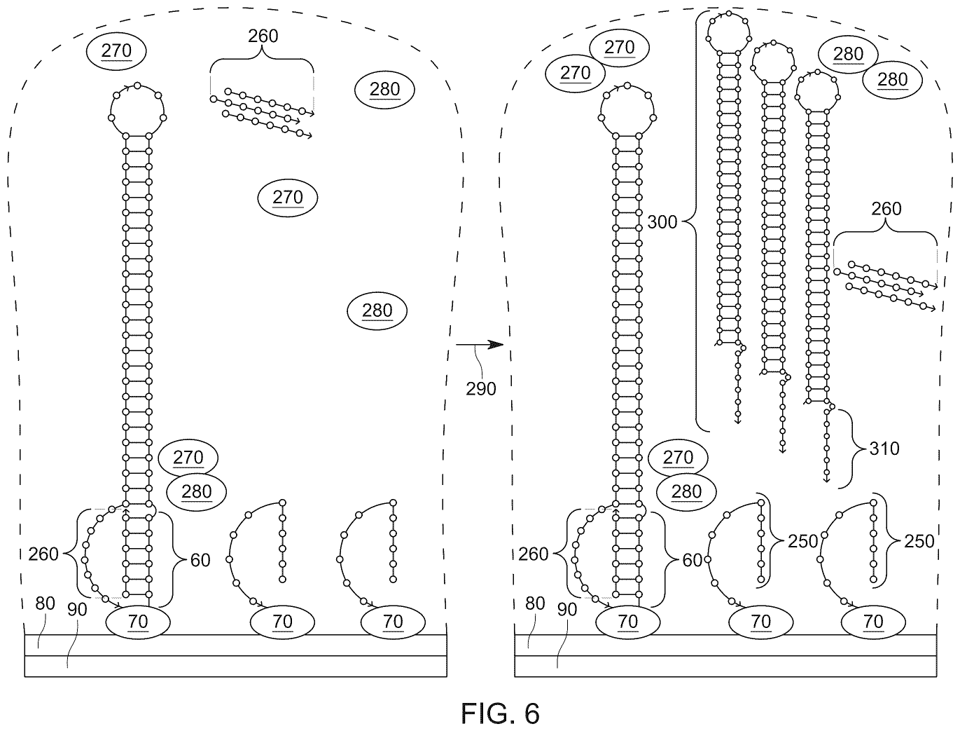

In a first method, shown in FIG. 5, the surface 80 of the chip 90 has two types of DNA constructs: fragments 170 and ssDNA “Grabbers” 250 (having, e.g., a length of 20 bp). The “grabbers” 250 are bound to the coated surface 80 by way of a DNA spacer 240 (having, e.g., a length of 20 bp) with internal DNA modification 70 (e.g., DBCO) on its 3′ end. A Strand Displacement Amplification (SDA) 290/Nicking Enzyme Amplification Reaction (NEAR) performed on the surface of the chip utilizes two enzymes: strand-displacing DNA polymerase 280, e.g. Bst, and nicking enzyme 270, e.g. BbvCI, as well as a primer 260 with a Nicking Endonuclease recognition and cleavage site having a length, e.g., of 20 bp. The primer 260 binds to the single stranded primer binding sequence 60 of the fragment 170. The nicking enzyme 270 creates a nick at a recognition site at the end of the primer 260, allowing the DNA polymerase 280 to copy over the template strand, while displacing any previously existing strands. This continuous isothermal reaction leads to a plethora of DNA hairpins 300 in liquid phase (i.e., Strand Displacement Amplification (SDA) Products), as shown in FIG. 6, that are identical to the solid phase fragments 170 except that they contain a ssDNA terminal sequence 310 that is complementary to the ssDNA “Grabber” sequence 250.

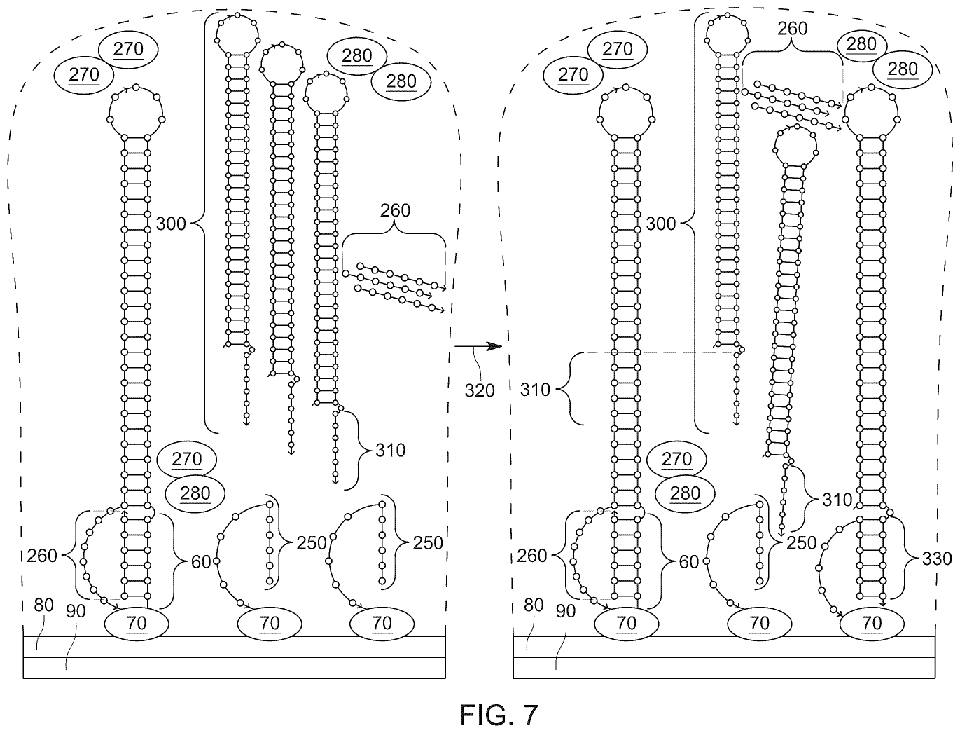

As shown in FIG. 7, the hairpin 300 terminal sequences 310 hybridize 320 to the grabber sequences 250.

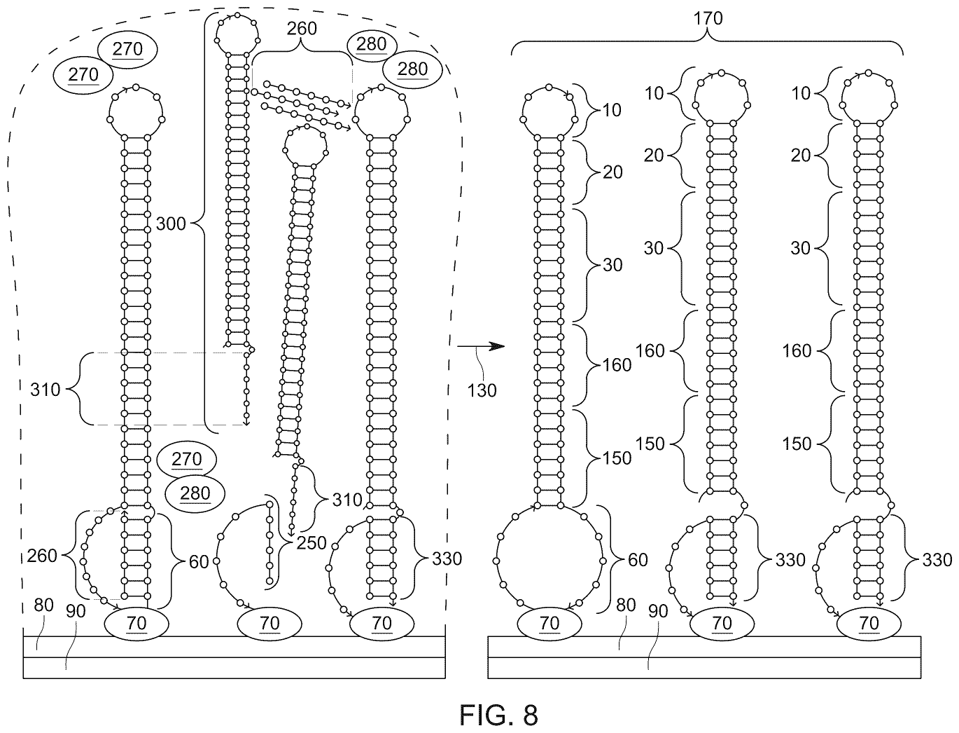

After washing 130 away the enzymes and primers, the original fragments and amplified fragments that are grabbed on the surface chip remain. See FIG. 8. The amplified fragments contain dsDNA 330, which is concatenated from the ssDNA “Grabber” sequence 250 and its complementary ssDNA 310.

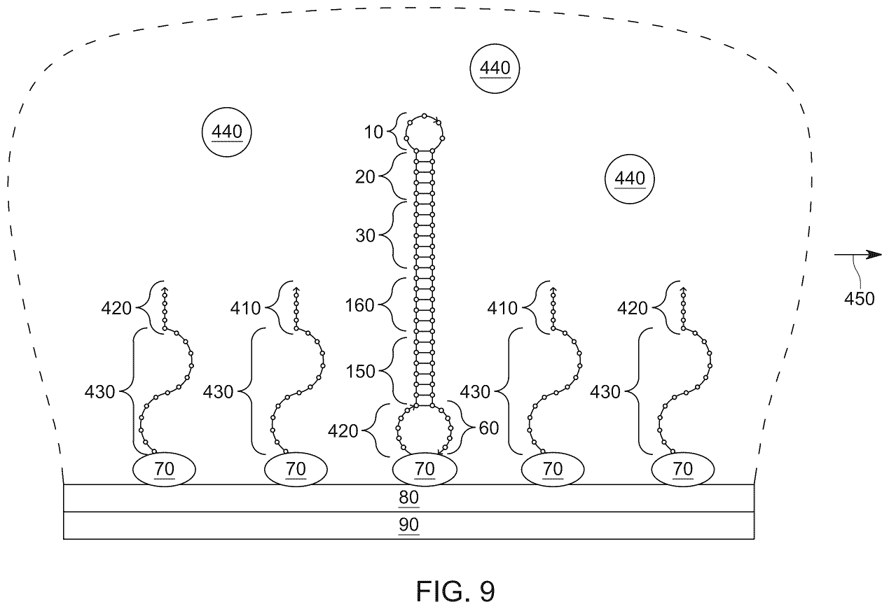

In a second (Melt) donor amplification method, the chip illustrated in FIG. 9 has three DNA constructs: fragments 170, solid-phase forward primers 410, and solid-phase reverse primers 420. The Forward Primer 410 is complementary to the single stranded primer binding site 60. The reverse primer 420 is complementary to the Reverse Priming site for Donor Amplification 400. The primers 410, 420 are each bound to an internal DNA modification 70 by way of a Long, Flexible Spacer 430. Similarly to Solid-Phase Polymerase Chain Reaction (SP-PCR), a thermostable DNA polymerase 440, e.g. Taq, is used in an on-chip reaction, thermocycled between three steps: annealing 450, extension 460, and denaturation 470.

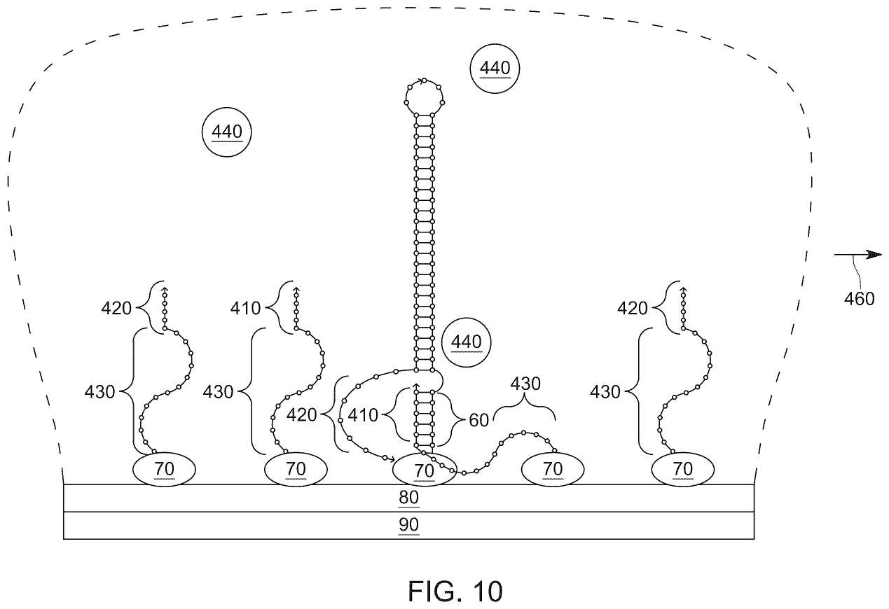

FIG. 10 shows the annealing step 450, in which the solid-phase primers 410, 420 bind to the single stranded sequence 60 at the base of the DNA fragments 170.

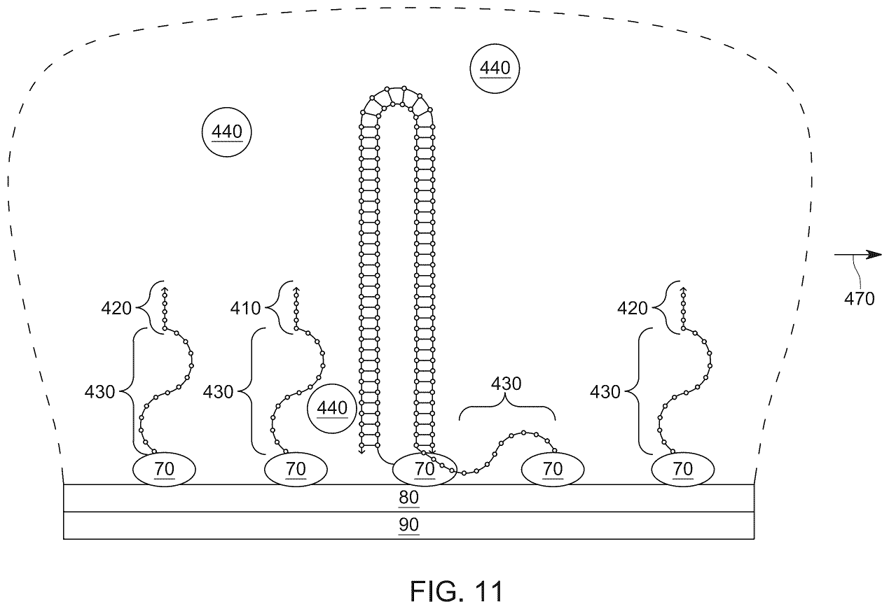

In the extension step 460, DNA polymerase 440 synthesizes a copy of the template strand. Importantly, the template strand and the coding strand are attached to the surface by two different DBCO groups 70. See FIG. 11.

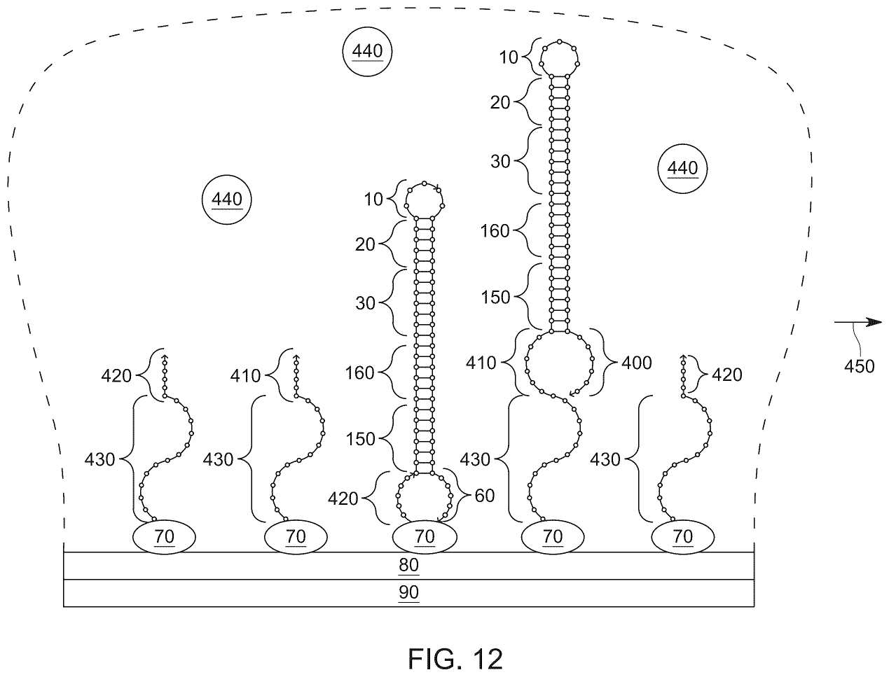

In the denaturation step 470 (see FIG. 12), each double stranded DNA is turned into a single stranded complex having a Reverse Priming site for Donor Amplification 400, with a length e.g., of 20 bp. Since all fragments are solid phase, this step leads to separation of all fragments. In other words, the extended double strand shown in FIG. 11 separates at a point along the sequence that results in two, surface bound, substantially identical double stranded stems with single stranded caps or hairpins. In the next cycles, these newly synthesized strands can be used as template strands for primers that are still on the surface, leading to exponential amplification.

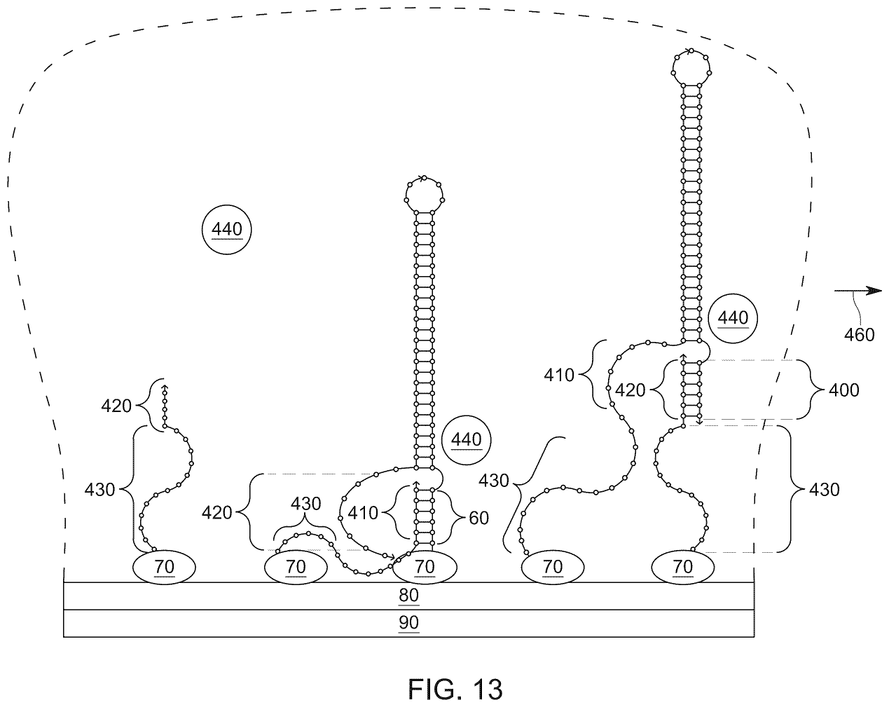

FIGS. 13-15 illustrate the iterative nature of the process in some embodiments.

The method of the present subject matter may be used to assemble longer sequences. For example (not illustrated), a product sequence 5′-ACGCCACGGACCAGTCTGACCGCTAGTACTCGACTG-3′ (k=16) may be produced using the acceptor 5′-ACGCCACGGACCAGTCTGAC-3′ and the donor 5′-TGACCGCTAGTACTCGACTG-3′.

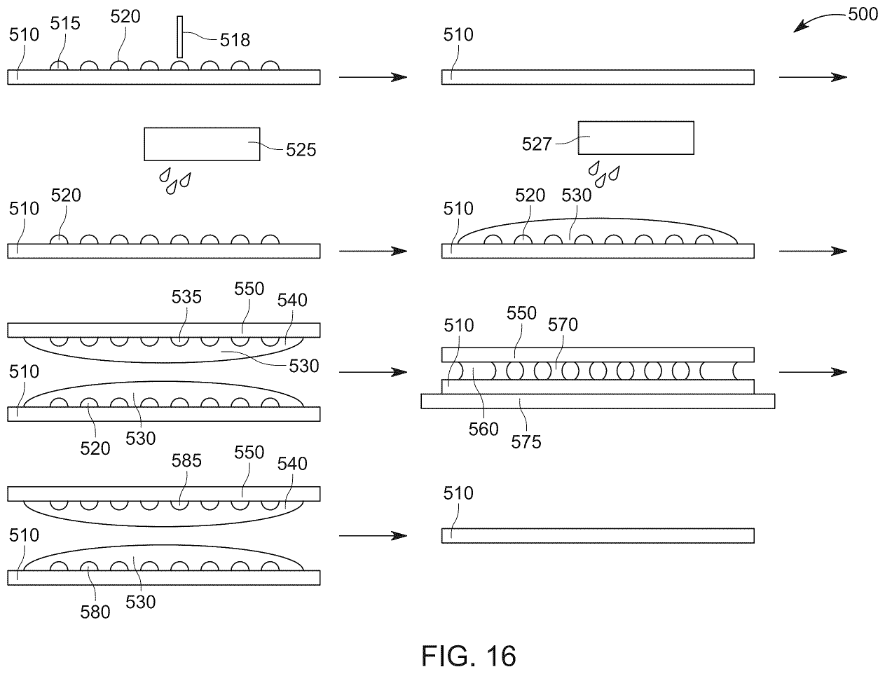

Turning next to FIG. 16, supporting hardware 500 in a system for the implementation of the biological processes described herein, according to an embodiment of the present subject matter, is illustrated. DNA-containing droplets 520 are placed onto a first substrate 510 (e.g., glass substrate with functional chemistry) by means of a spotting nib or other high-throughput dispense tool 518 such as an acoustic arrayer (e.g. Echo Liquid Handler, available from Beckman Coulter® Life Sciences). The substrate 510 retains the DNA fragments 515 and after the DNA fragments are bound, the droplets 520 may be washed off. A second substrate 550 is concurrently populated with droplets 535 containing the appropriate enzymatic reagents for subsequent reactions using dispense tool, e.g. an inkjet head 525. A barrier layer 530, 540, e.g., oil, may be added to both substrates to mitigate dry-out, using said dispense tool 525 or another dispense mechanism 527, e.g. an inkjet head. The substrates are then placed in alignment such that the droplets 520, 535 are precisely in line in complementary pairs. The substrates are subsequently brought together to form droplets 570 containing mixtures of the droplets' respective reagents. A barrier oil layer may mix as well to form a combined barrier layer 560 as a mitigant for aqueous droplet dry out. The reactions are heated to suitable reaction temperatures with a contact heater or heating chamber 575. The machine 575 may also provide humidity control, producing humidification during this time to further reduce the likelihood of droplet evaporation. Following the completion of the reactions the substrates 510, 550 are separated and washed. Post separation, droplet 580 remains on substrate 510 and droplet 585 remains on substrate 550.

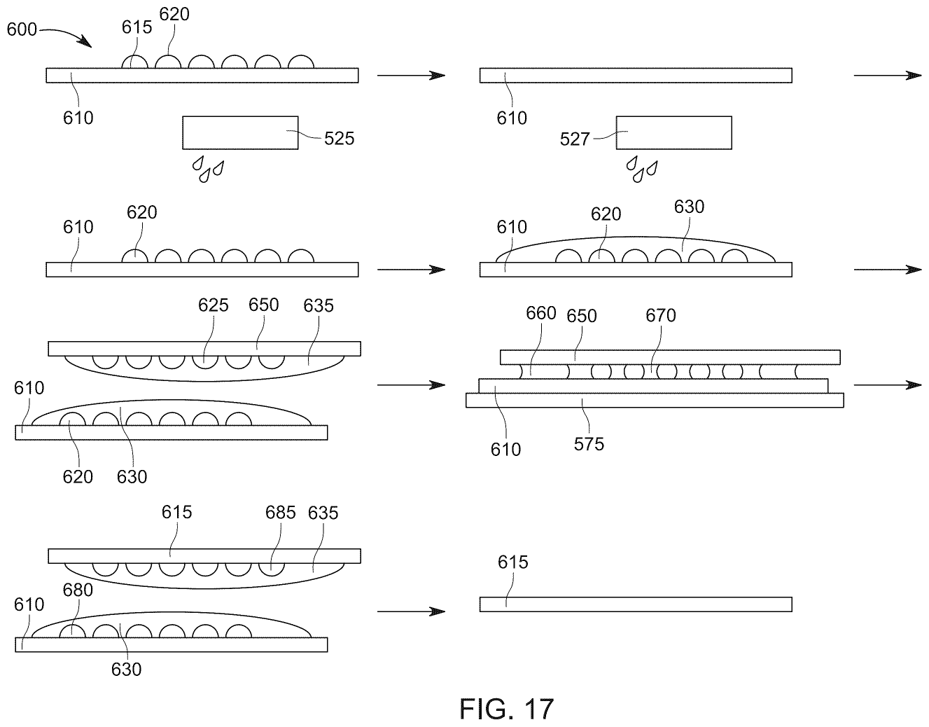

FIG. 17 illustrates Alignment and ligation of a higher order assembly using an embodiment of supporting hardware necessary for the implementation of the biological processes described here-in. The substrate 610 retains droplets 620 containing the DNA fragments 615. After the DNA fragments 615 are bound, the droplets 620 may be washed off. A second substrate 650 is concurrently populated with droplets 625 containing the appropriate enzymatic reagents for subsequent reactions using dispense tool 525, e.g. an inkjet head. Barrier layers 630, 635, e.g., oil, preventing dehydration are subsequently added to both substrates using said dispense tool 527. Alternatively, substrates may be situated in a humidity chamber obviating the need for said barrier layers. The substrates are then placed in alignment such that the droplets 620, 625 are precisely in line in complementary pairs. The substrates 610, 650 are subsequently brought together to form droplets 670 containing mixtures of the droplets' 620, 625 respective reagents. The barrier oil layers 630, 635 may mix as well to form a combined barrier layer 660 as a mitigant for aqueous droplet dry out. The reactions are heated to suitable reaction temperatures with a heater 575; during this time there may also be humidification to further reduce the likelihood of droplet evaporation. Following the completion of the reactions, the substrates 610, 650 are separated, such that the first substrate 610 has droplets 680 and the second substrate 650 has droplets 685 and washed.

### Dry-Out Reduction

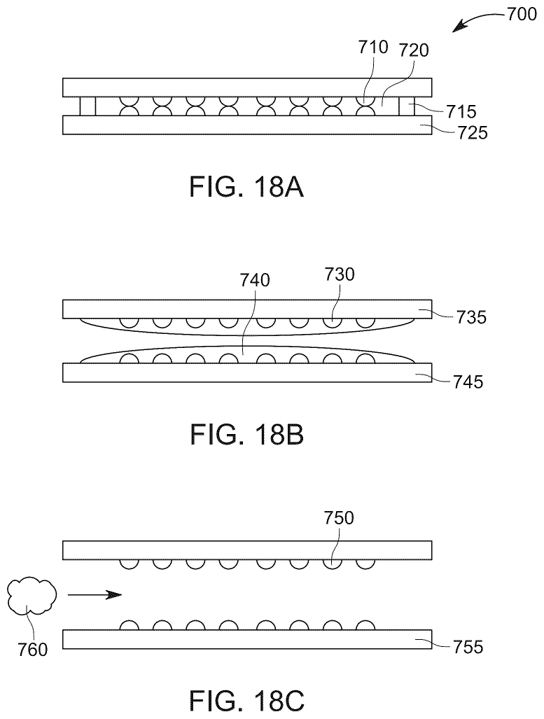

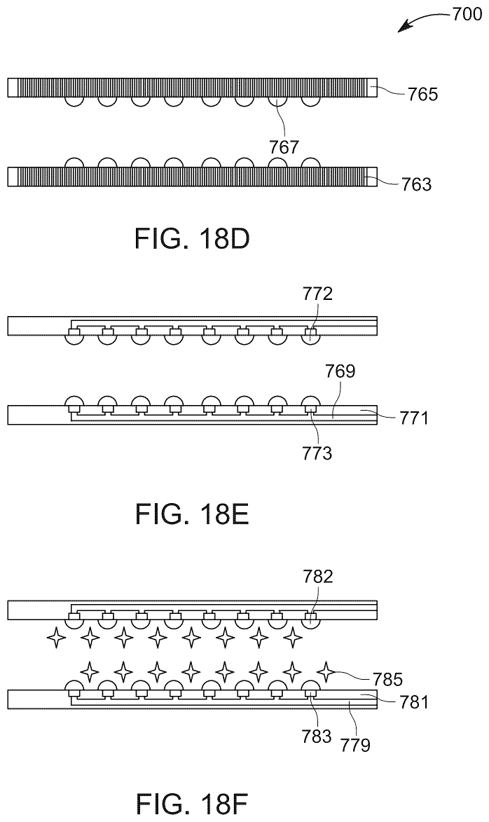

System configurations for various methods of reducing droplet dry-out are described in FIGS. 18A, 18B, 18C, 18D, 18E, and 18F. FIGS. 18D-18F illustrate engineered glass slides having features designed to mitigate droplet dry-out.

FIG. 18A illustrates a reaction chamber 700 for use with a fully constrained method whereby a seal 715, e.g., formed of an elastomer, is formed around the aqueous reagent droplets 710 and is compressed between the two functionally active slides 725 (e.g., glass slides). The formed chamber may be filled with a pressurized gas or with liquid barrier layer 720 that reduces the rate of evaporation of the droplets contained therein. This technique may be used to surround a full array, part of an array, or singulated droplet chambers.

FIG. 18B demonstrates an unconstrained method of mitigating droplet evaporation. The aqueous reagent droplets 730 are protected from exposure to ambient air by an oil barrier layer 740. The barrier layer 740 of the slides 745 (e.g., glass) is retained on the surface by interfacial forces between the surface functionalization 735 and the compound(s) present in the oil 740.

FIG. 18C illustrates humidity 760 control, both within the machine to ensure the aqueous reagent droplets 750 are protected from exposure to ambient air conditions that may accelerate the dry out of the droplets. The humidity 760 control can take place both within the slides 755 and/or within the machine and, in some cases, within the array.

As shown in FIG. 18D, the reaction chamber 700 may include porous glass slides 765 having nano capillaries 763 that enable humid air to enter the space between the slides 765 while preventing water or other liquid phase reagents from the aqueous droplets 767 from passing through.

FIG. 18E depicts glass slides 771 with microfluidic (or nanofluidic) channels etched into the substrate, such that each fluidic channel 769 feeds a single aqueous droplet 772 from a common reservoir. An ultrafilter 773 or positive pressure membrane may be fitted between the common reservoir and the droplets 772 to prevent cross-contamination between droplets from DNA or other molecules.

Similarly, FIG. 18F depicts a system with an ionic membrane 783 in place of the ultrafilter 773, operated by modulating the electric charge of the membrane (i.e., using an electronic trigger 785). The ionic membrane 783 can regulate flow of DNA or other molecules to or from the aqueous droplets 782 through a fluidic channel 779 in the glass slide 781 on demand, using a standard multiplexing technique.

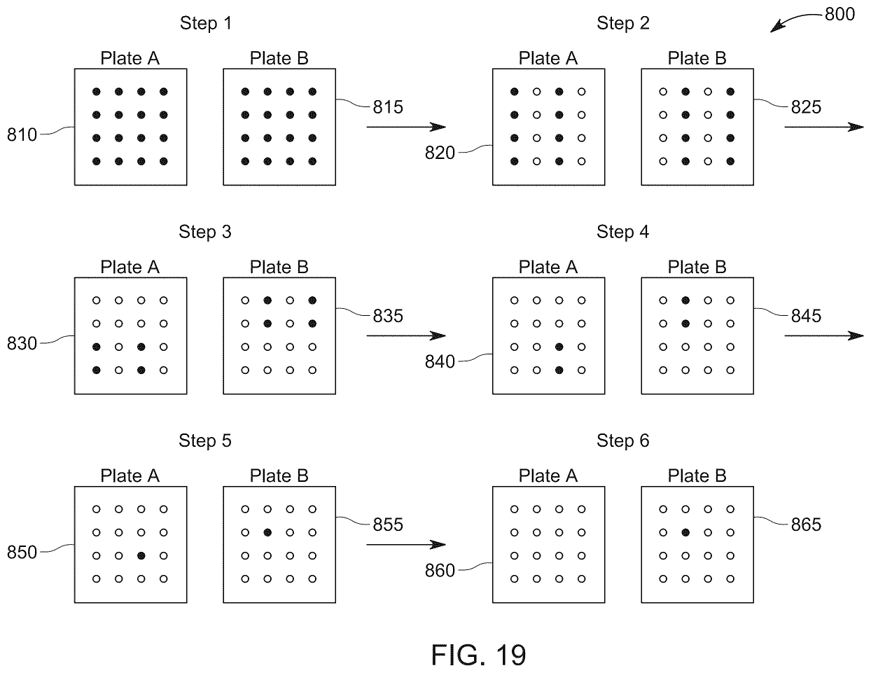

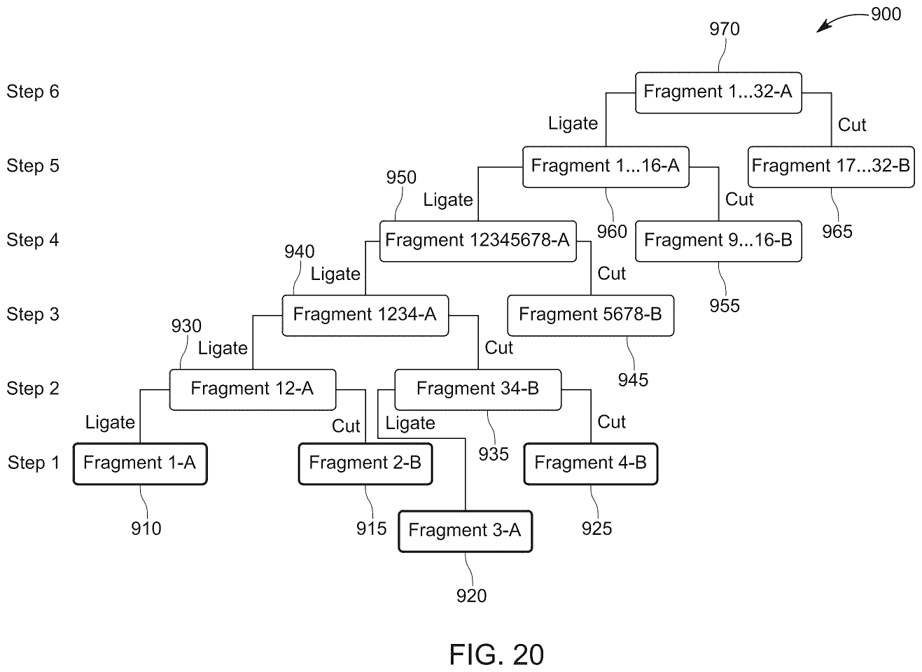

To form a full sequence 970, gene fragments are assembled hierarchically using a 2-plate system, an example of which is shown in FIG. 19. The two plates are labeled Plate A 810, 820, 830, 840, 850, 860 and Plate B 815, 825, 835, 845, 855, 865. FIG. 19 illustrates a method having Steps 1-6, although the method disclosed herein is not limited with respect to the number of steps. In Step 1, the initial fragments are spotted onto plates 810, 815. The spots are targeted using a dispense mechanism with reagents that either cut or ligate gene fragments 910, 915, 920, 925 from one plate to the next, as illustrated in FIG. 20. FIG. 19 illustrates a sample pattern in Step 2, in which Plate A 820 and Plate B 825 each have ligated fragments bound in the shaded circles, e.g. 930, 935, and cut fragments in the white circles, e.g., 930, 935. This process is repeated through a series of steps with either Plate A or Plate B serving as a donor for a first set of spots and simultaneously serving as a receiver for a second set of spots, gradually assembling gene fragments 940, 945, 950, 955, 960, 965. The process terminates with final assembly of a full gene 970 on Plate B 865. This system is agnostic of the number of fragments needed for a target gene as a spot can be filled with reagent on an as-needed basis. In the instance where multiple unique sequences are assembled in parallel, not all steps will involve all target sequences. There is no limit to the number of parallel sequences that may be assembled. In this example, 32 distinct gene fragments have been assembled. Each fragment contains a short sequence of 8 nucleotides at minimum, with the addition of sequence recognition sites detailed elsewhere in this disclosure. The 8-nucleotide sequence contains a 4-nucleotide sticky end and a 4-nucleotide sequence that will serve as a sticky-end in a subsequent assembly step. As a consequence, the minimum part library required to assemble every possible fragment will be 4⁸ parts, or 65,536 unique segments.

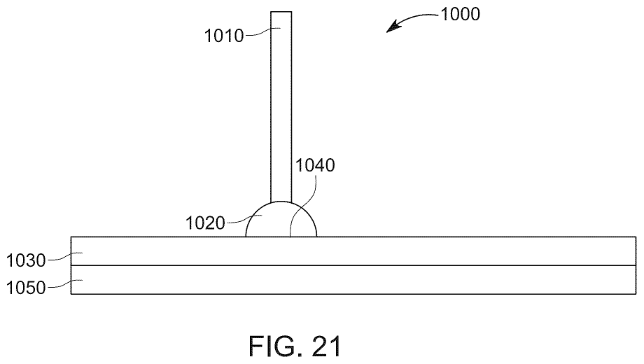

FIG. 21 demonstrates a harvesting technique 1000 performed on a glass substrate 1030 with assembled DNA bound to its surface at spot 1040. A droplet 1020 containing a cutting restriction enzyme is deposited coaxially with the center point of a DNA bound spot 1040 using a precision dispense/aspirate mechanism 1010, such as a servo controlled pL syringe. The heater block 1050 subsequently heats the spot 1040 to encourage the restriction enzymes to cut the fragments at the base where they connect to the substrate 1030. As the droplet is heated during this process, the dispense mechanism 1010 may continuously deposit additional solution to prevent over-concentration of the restriction enzyme and to minimize star activity. Following restriction, the droplet is aspirated into the syringe 1010 and may be transferred to a well plate or a single use capped tube.

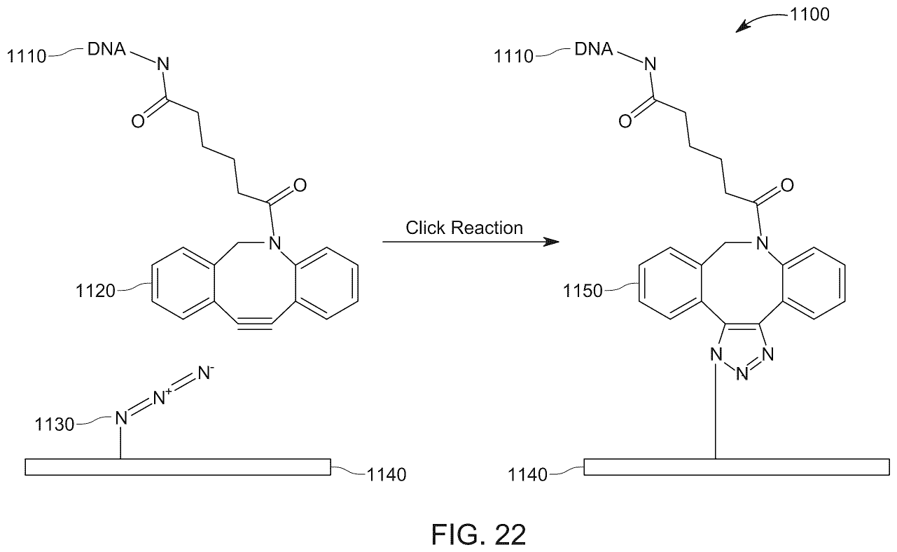

FIG. 22 reflects the reaction chemistry used to bind DNA fragments 1110 to the surface 1140 of a glass slide or plate, e.g., a borosilicate glass substrate. The reaction chemistry has been described elsewhere as copper-free Click chemistry. The DNA fragments may be bound to a Dibenzocyclooctyl (DBCO) group at the 5′ or 3′ end and are provided as an input to the process. Similarly, the substrates 1140 are covalently bound with Azide groups 1130 prior to process initiation. Reagents containing the DBCO-DNA functional group 1120 fragments are deposited to the glass substrate 1140, and the DBCO-DNA exclusively reacts with the Azide 1130 bound surface 1140 to form triazoles 1150 thereby specifically tethering the DNA to the substrate.

In this patent specification, novel polynucleotide synthesis systems and methods are described. While the present subject matter is described with reference to exemplary embodiments, the present subject matter is not limited to these embodiments. On the contrary, many alternatives, changes, and/or modifications shall become apparent to a person of ordinary skill in the art (“POSITA”) after this patent specification has been reviewed along with its accompanying illustrations. Accordingly, all such alternatives, changes, and modifications are to be treated as forming part of the present subject matter.

It should be understood, of course, that the foregoing relates to exemplary embodiments of the invention and that modifications may be made without departing from the spirit and scope of the invention as set forth in the following claims.

What is claimed is:

1. A method of assembling polynucleotides, comprising:
providing a reaction apparatus including a first substrate (90) with a first surface (80) having an acceptor deoxyribonucleic acid (DNA) fragment (1) bound thereon at points in an array, and a second substrate (90) with a second surface (80) having a donor DNA fragment (170) bound thereon at points in a corresponding array, with the first surface (80) aligned against the second surface (80) such that the first and second substrates (90) form a reaction volume therebetween;
applying an enzyme mixture (100, 180, 190) as droplets to the first surface and the second surface (80), thereby:
cleaving (110) the acceptor and donor DNA fragments (1, 170) at an enzyme recognition site (30, 50, 150) with the enzyme mixture (100, 180, 190) to liberate at least one donor sequence (160) having a first single strand free end (210) and to expose at least one acceptor sequence (40) having a second single strand free end (120) that is complementary to the first single strand free end (210); and
ligating (200) the first single strand free end (210) to the second single strand free end (120) to form an intermediate DNA fragment (1) having a sequence (230) concatenated from the donor sequence (160) and the acceptor sequence (40), wherein the intermediate DNA fragment (1) is bound to the first surface or the second surface (80); and
washing (130) the first surface (80) and the second surface (80) to remove the enzyme mixture (100, 180, 190) and unbound byproducts (10, 20, 30, 220).

2. The method of claim 1, wherein the acceptor DNA fragment and the donor DNA fragment (1, 170) are selected from a library of presequenced oligonucleotides.

3. The method of claim 1, further comprising disabling misligated and/or capping uncapped DNA fragments before or after washing.

4. The method of claim 1, wherein the enzyme mixture comprises restriction enzymes (100, 180) and DNA ligase (190), deposited as a droplet at each of the points.

5. The method of claim 1, wherein the first substrate (90) simultaneously serves as a donor for a first subset of the first array and as an acceptor for a second subset of the first array.

6. The method of claim 1, further comprising amplifying the donor sequence (160) by:
providing the substrate (90) having bound thereto: the donor DNA fragment (170) containing a donor sequence (160) and a primer binding site (60), a solid-phase forward primer (410) having a complementary sequence to the primer binding site (60); and a solid-phase reverse primer (420);
applying a thermostable DNA polymerase (440) to the substrate (90); and
thermocycling between:
annealing (450) the solid-phase forward primer (410) and the solid-phase reverse primer (420) to the primer binding site (60);
extending (460) the donor DNA fragment (170) by synthesizing a copy of the donor sequence (160); and
denaturing (470) the extended DNA fragment to form two single stranded segments bound to the substrate (90).

7. The method of claim 6, wherein the solid-phase forward primer (410) and the solid-phase reverse primer (420) are bound to the substrate (90) by way of a spacer (430).

8. The method of claim 1, further comprising moving the first substrate (810, 820, 830, 840, 850, 860) relative to the second substrate (815, 825, 835, 845, 855, 865) such that the points of the first array align with the points of the corresponding array in a predetermined order.

9. The method of claim 1, further comprising amplifying the donor sequence (160) by:
providing a surface (80) having bound thereto the donor DNA fragment (170) containing a top loop (10), a stem (20), a donor sequence (160), and a restriction recognition site (30, 150); and at least one single stranded DNA (ssDNA) “grabber” (250);
depositing an enzyme composition on the surface, the enzyme composition comprising strand-displacing DNA polymerase (280) and a nicking enzyme (270) and a primer (260) having a sequence containing a nicking endonuclease recognition and cleavage site, thereby:
binding the primer (260) to the donor DNA fragment (170);
nicking the nicking endonuclease recognition and cleavage site;
producing a strand displacement amplification product (300) having a donor sequence (160) identical to the donor DNA fragment (170); and
hybridizing (320) the strand displacement amplification product (300) to the ssDNA “grabber” (250) to form an amplified fragment having a sequence (330) concatenated from the ssDNA “grabber” (250) and a ssDNA sequence (310) complementary to the ssDNA “grabber” sequence; and
washing (130) away the strand-displacing DNA polymerase (280), the nicking enzyme (270), the primer (260), and any unbound strand displacement amplification product (300).

10. The method of claim 1, further comprising reducing evaporation of a solvent.by applying a barrier layer (530) to the first surface (510) and the second surface (550).

11. The method of claim 1, further comprising reducing evaporation of a solvent by retaining a pressure in the reaction volume greater than 1 atmosphere gauge.

12. The method of claim 1, further comprising harvesting (1000) the DNA fragment by applying a cutting enzyme to liberate the DNA fragment from the first surface or the second surface (1030) and aspirating the DNA fragment.

13. A nucleotide assembly reaction apparatus, comprising:
a first substrate (510) having a first planar surface with a first array of reaction spots (515) operative to bind an individual nucleotide chain; and
a second substrate (550) having a second planar surface with a second array of reaction spots operative to bind an individual nucleotide chain, having a reaction position facing the first planar surface;
wherein the first planar surface and the second planar surface form a plurality of individually addressable, reversibly enclosable reaction chambers (570) therebetween.

14. The nucleotide assembly reaction apparatus of claim 13, further comprising a deposition tool (518, 525, 527) selected from the group consisting of a print head, a syringe, and a single-droplet ejector.

15. The nucleotide assembly reaction apparatus of claim 13, further comprising a harvesting tool (1010).

16. The nucleotide assembly reaction apparatus of claim 13, wherein the first planar surface and the second planar surface comprise a coating containing azide functional groups (1130).

17. The nucleotide assembly reaction apparatus of claim 13, further comprising a seal (715) surrounding the plurality of reaction chambers (570).

18. The nucleotide assembly reaction apparatus of claim 13, further comprising a heater (575) operative to control a temperature in the plurality of reaction chambers (570).

19. The nucleotide assembly reaction apparatus of claim 13, further comprising a humidity control system (760).

20. The nucleotide assembly reaction apparatus of claim 13, wherein at least one of the first substrate and the second substrate (765) is porous (763) and operative to conduct humid air.

21. The nucleotide assembly reaction apparatus of claim 13, wherein at least one of the first substrate and the second substrate (771) has at least one fluidic channel (769) etched therethrough, wherein the fluidic channel (769) is operative to conduct moisture to one or more of the reaction spots.

22. The nucleotide assembly reaction apparatus of claim 13, further comprising a membrane or filter (773, 783) operative to prevent passage of nucleotides.

23. The nucleotide assembly reaction apparatus of claim 13, further comprising a library of pre-sequenced oligonucleotides, wherein the first substrate and the second substrate have the pre-sequenced oligonucleotides selected from the library coupled thereto.
